(19) United States
(12) Patent Application Publication
(10) Pub. No.: US 2019/0262165 A1
(43) Pub. Date: Aug. 29, 2019

(54) SELF-ADJUSTING TOOL FOR CORRECTING INGROWN TOENAIL

(71) Applicant: 32905.36 PATENT APPLICATION TRUST, Alexandria, VA (US)

(72) Inventor: ABDULRAHMAN MAHMOUD ABDULLAH AL SUWAIDI, DOHA (QA)

(21) Appl. No.: 16/332,784

(22) PCT Filed: Jan. 23, 2018
(86) PCT No.: PCT/US2018/014833

§ 371 Date: Mar. 12, 2019

Related U.S. Application Data
(60) Provisional application No. 62/511,325, filed on May 25, 2017.

(51) Int. Cl. A61F 5/11

(52) U.S. Cl. CPC A61F 5/11; A45D 29/00

(57) ABSTRACT

The self-adjusting tool (10) for correcting ingrown toenail uses self-adjusting elastic tension to lift the ingrown ends (N1, N2) of an ingrown toenail (N). In a first embodiment, the self-adjusting tool (10) includes two identical leaf springs (12a, 12b), each having a flat end (16a, 16b) and a band (14a, 14b) forming a flat loop at the opposite end. The two springs (12a, 12b) are interlocked by sliding the flat end (16a, 16b) of one spring through the band (14a, 14b) of the other, and the free ends (16a, 16b) are glued to lateral edges of the ingrown toenail (N) to flatten the toenail (N) by self-adjusting elastic tension. Other embodiments show at least one resilient member flexed across the ingrown toenail and anchored at opposite lateral edges on the ingrown toenail to pull the ingrown edges of the toenail away from engagement with the toe.

### TECHNICAL FIELD

The disclosure of the present patent application relates generally to podiatry, and particularly to various embodiments of a self-adjusting tool for correcting ingrown toenail that use self-adjusting elastic tension to lift the ingrown lateral edges of an ingrown toenail.

### BACKGROUND ART

An ingrown toenail (also known as onychocryptosis or unguis incarnates), or a deformed toenail, is a common form of nail disease. It is an often painful condition in which the toenail grows so that it cuts into one or both sides of the nail bed (or paronychium). Mild to moderate cases are often treated conservatively with warm water and epsom salt soaks, antibacterial ointment and the use of dental floss. If conservative treatment of a minor ingrown toenail does not succeed, or if the ingrown toenail is severe, surgical treatment may be required. A so-called “gutter splint” may also be improvised by slicing a cotton-tipped wooden applicator diagonally to form a bevel and using this to insert a wisp of cotton from the applicator head under the nail to lift it from the underlying skin after a foot soak.

For more severe cases, surgical treatment may be carried out by a podiatrist. This is typically an in-office procedure requiring local anesthesia and special surgical instruments. The surgical approach is the removal of the offending part of the nail plate, referred to as a wedge resection. If the ingrown toenail recurs despite this treatment, destruction of the sides of the nail with chemicals or excision is performed, a procedure known as a matrixcestomy. Although surgical treatment for ingrown nails is more effective at preventing the nail from regrowing inwards as compared to non-surgical treatments, the pain, healing time and inconvenience to the patient must be considered. Thus, a self-adjusting tool for correcting ingrown toenail solving the aforementioned problems is desired.

### DISCLOSURE OF INVENTION

The self-adjusting tool for correcting ingrown toenail uses elastic self-adjusting tension to lift the ingrown lateral edges of an ingrown toenail and to correct deformation of the toenail at the root base. In a first embodiment, the tool includes two identical leaf springs, each having a flat end and a band forming a flat loop at the opposite end. The two springs are interlocked by sliding the flat end of one spring through the band of the other, and the free ends are glued to lateral edges of the ingrown toenail to lift and flatten the toenail by elastic tension. As the toenail flattens, the leaf springs self-adjust or slide toward each other.

In a second embodiment, two flat loop bands are glued to opposite lateral edges of the ingrown toenail and a leaf spring of suitable length has opposite ends inserted into the bands to raise the lateral edges by elastic, contracting tension. In a third embodiment, the bands are replaced by annular sleeves and the flat leaf spring is replaced by a spring steel rod. Multiples rods may be placed in parallel. In a fourth embodiment, a flat loop band is glued to a lateral edge of the ingrown toenail and the leaf spring is T-shaped, having the end of elongate shaft inserted into the band and the crossbar of the T-shaped head glued to the opposite lateral edge of the toenail to flatten the toenail by elastic, retrievable tension.

These and other features of the present disclosure will become readily apparent upon further review of the following specification and drawings.

### BRIEF DESCRIPTION OF THE DRAWINGS

FIG. 1 is an environmental front view of a first embodiment of a tool for correcting an ingrown toenail.

FIG. 2A is a perspective view of the tool of FIG. 1.

FIG. 2B is an exploded front view of the tool of FIG. 1.

FIGS. 3A and 3B illustrate successive method steps for the application and use of the tool of FIG. 1.

FIG. 4A is a side view of a second embodiment of a tool for correcting an ingrown toenail.

FIG. 4B is a perspective view of the tool of FIG. 4A.

FIG. 5A is an exploded perspective view of a third embodiment of the ingrown toenail correcting tool.

FIG. 5B is a perspective view of the tool of FIG. 5A.

FIG. 5C is a front view of the tool of FIG. 5A.

FIG. 6A is an exploded perspective view of a fourth embodiment of a tool for correcting an ingrown toenail.

FIG. 6B is a perspective view of the tool of FIG. 6A, shown under flexure.

Similar reference characters denote corresponding features consistently throughout the attached drawings.

### BEST MODE(S) FOR CARRYING OUT THE INVENTION

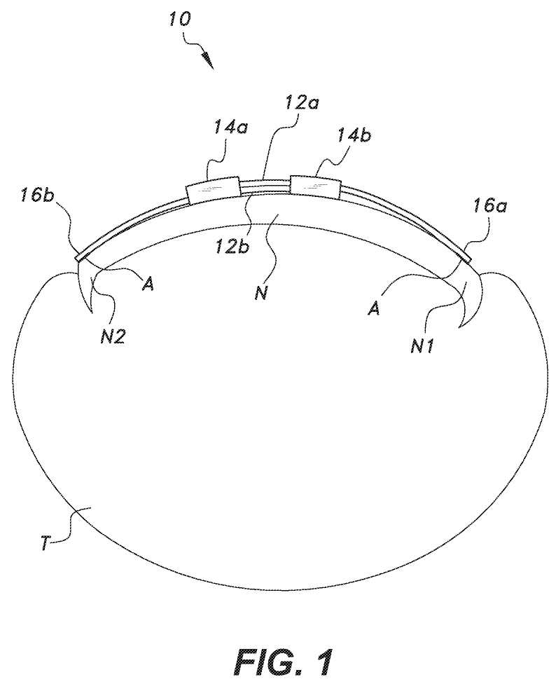

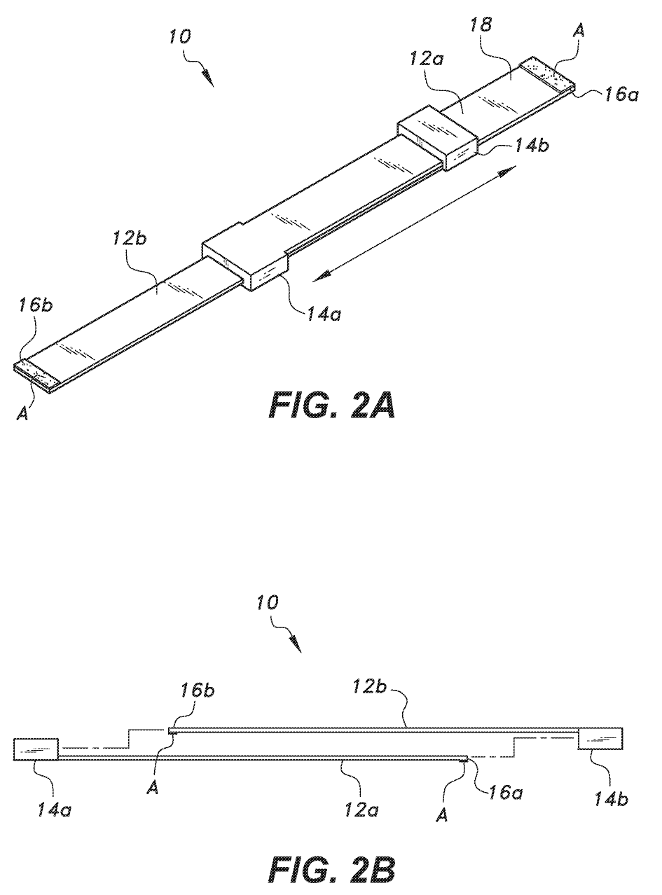

The self-adjusting tool 10 for correcting ingrown toenail uses elastic, self-adjusting tension to lift the ingrown lateral edges of an ingrown toenail N to flatten the toenail. As shown in FIGS. 1, 2A and 2B, a first embodiment of the self-adjusting tool 10 includes two leaf springs 12a, 12b, each of the leaf springs 12a, 12b having a corresponding band 14a, 14b at one end and a flat free end 16a, 16b at the opposite end. The bands 14a, 14b form a flat loop. The free end 16a of spring 12a is slidable through the band 14b of spring 12b, and the free end 16b of spring 12b is slidable through the band 14a of spring 12a so that the leaf springs 12a, 12b are slidably interlocked and the total combined length of the self-adjusting tool 10 can be adjusted initially by sliding the springs 12a, 12b towards and away from each other. As such, the self-adjusting tool 10 can be adapted for use on toenails of various sizes.

Once adjusted to the proper length, the free ends 16a, 16b can be fixed to the lateral edges of the ingrown toenail N by a layer of adhesive A. The leaf springs 12a, 12b bear against the middle of the toenail N, and the resiliency of the springs 12a, 12b urges the free ends 16a, 16b to pull the curved or curled lateral edge(s) of the ingrown toenail N upward to flatten the toenail N and relieve pressure of the curled nail edges pressing into the toe T. As the toenail N is lifted, the leaf springs 12a and 12b slide toward each other, thereby adjusting the total length of the tool 10. The sliding action of one leaf spring with respect to the other as the toenail N slowly flattens allows the self-adjusting tool 10 to self-adjust in length and configuration, e.g., from a curved configuration to a linear configuration.

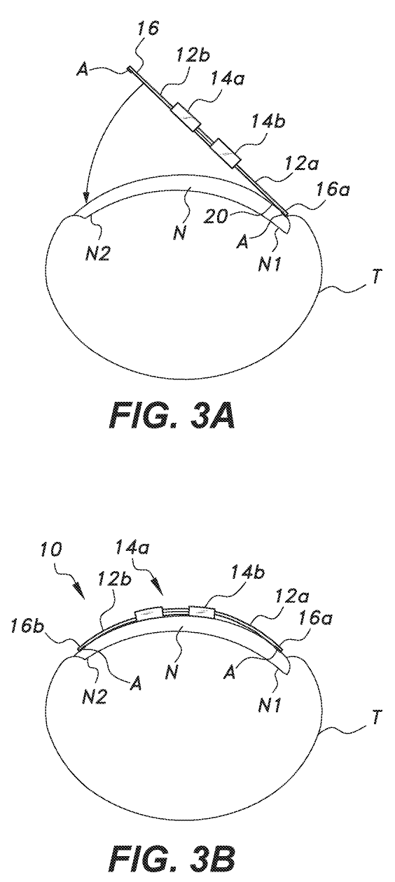

In use, a thick ingrown toenail may need to be thinned by scraping the toenail N until the toenail is soft, and/or chopping off part of an excessively long toenail N. After that, FIG. 3A shows the free end 16a of spring 12a being attached to an edge N1 of the ingrown toenail N by a layer of adhesive A. FIG. 3B shows the curvature of the leaf springs 12a, 12b after the free end 16b of spring 12b has been attached to the opposite edge N2 of the toenail N. The tool 10 may be used to correct a toenail N that is ingrown on both sides, or that is ingrown on one side only. It is anticipated that the tool 10 will flatten the toenail N in a period of 48 hours, but the self-adjusting tool 10 will be left in place for an additional period of time, e.g., two weeks, to facilitate complete correction of the defective nail. Additionally, follow-up treatment with antibiotics and foot lavage may be necessary for one or two months to prevent infection and permit proper healing of any scars from inward growth of the toenail N into the flesh of the toe T. It should be noted that the bands 14a, 14b permit the leaf springs 12a, 12b to be slidably connected to each other and, thereby, facilitate self-adjustment of a length of the tool 10 as the toenail N is flattened. In other words, the progressive flattening of toenail N results in a reduced overall length of self-adjusting tool 10, as the leaf springs 12a, 12b slide toward one another. The self-adjusting tool 10 for correcting an ingrown toenail can be disposed after a single use.

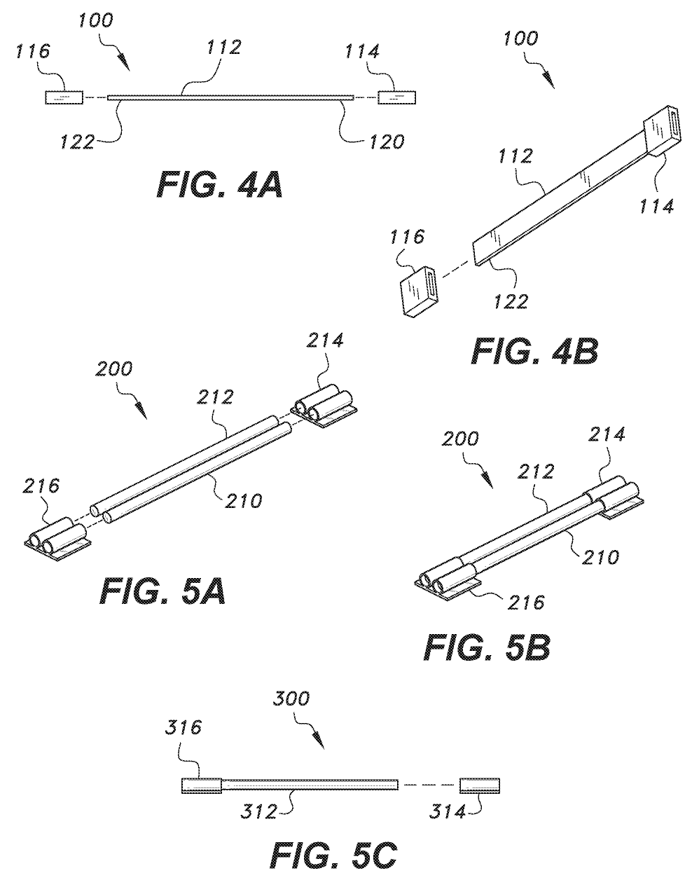

In the alternative embodiment of FIGS. 4A and 4B, the tool 100 includes a leaf spring 112 and both first and second bands 114, 116, respectively. In this embodiment, the first band 114 is adhered to the first side N1 of the ingrown toenail N, and the second band 116 is adhered to the second side N2 of the ingrown toenail N. The first end 120 of a leaf spring 112 of suitable length is slidably inserted through the first band 114, and the second end 122 of the leaf spring 112 is slidably inserted through the second band 116. In this embodiment, the leaf spring 112 may be a simple one-piece leaf spring, as shown. The resiliency of the leaf spring 112 urges the opposing ends 120, 122 upward so that the spring 112 returns to a linear configuration, thereby flattening the curled edges of the ingrown toenail N. While FIGS. 4A and 4B show first end 120 and second end 122 retained by bands 114 and 116 respectively, it should be understood that the first band 114 and the second band 116 are configured to receive additional portions of the leaf spring 112 as the leaf spring 112 returns to a linear configuration.

In the embodiment of FIGS. 5A and 5B, the tool 200 includes a spring formed as at least one elastic rod 210 or 212, the ends of which are slidably received by a corresponding cylindrical sleeve 214, 216. Although two rods 210, 212 are shown in FIGS. 5A, 5B, it will be understood that only a single rod may be necessary, depending on the application, as shown in FIG. 5C. In FIG. 5C, for example, the ingrown toenail correcting tool 300 has only a single elastic rod 312, with its ends being slidably received, respectively, by first and second sleeves 314, 316. The tools 200, 300 of FIGS. 5A-5C may also be used for correcting ingrown fingernails, where the rod(s) may be more aesthetically acceptable than the flat leaf springs of FIGS. 1-4B.

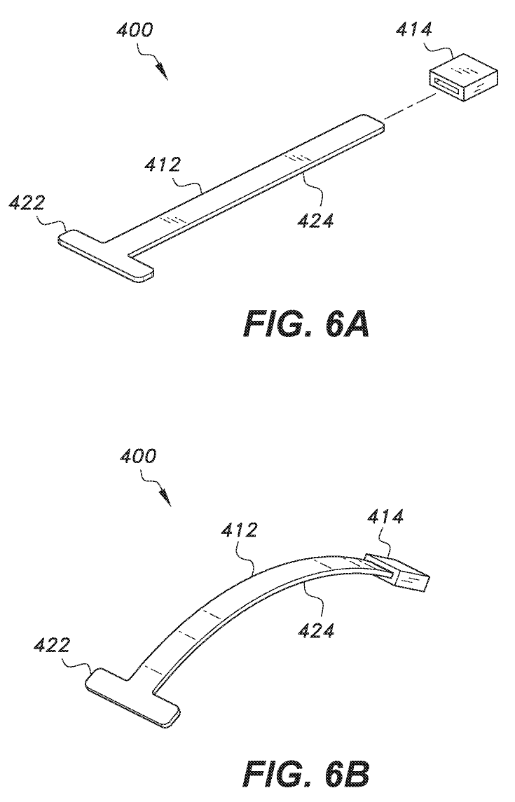

In the further alternative embodiment of FIGS. 6A and 6B, the leaf spring 412 of tool 400 is substantially T-shaped, as shown. In this embodiment, the leaf spring 412 is T-shaped, having an elongated shaft 424 capped by a crossbar 422. A flat band or loop 414 is attached to one edge of the ingrown toenail by adhesive, and the end of the shaft 424 is shortened, if necessary, and slidably inserted through the band 414. The leaf spring 412 is then bent across the toenail and the crossbar 422 is adhered to the opposite lateral edge of the toenail. The resiliency of the leaf spring 412 urges opposite ends of the spring 412 upward, thereby flattening the curled, ingrown edges of the toenail.

It is to be understood that the self-adjusting tool for correcting ingrown toenail is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A self-adjusting tool for correcting an ingrown toenail, comprising:
at least one elongate spring element adapted for extending across the ingrown toenail; and
means for securing opposing ends of the at least one spring element to opposing lateral edges of the ingrown toenail;
whereby resiliency of the at least one spring element raises an ingrown lateral edge of the toenail to pull an ingrown edge of the toenail away from the toe to correct the ingrown toenail.

2. The self-adjusting tool for correcting an ingrown toenail according to claim 1, wherein:
said at least one spring element comprises a pair of leaf springs, each of the leaf springs having a flat free end and an opposing end having a band defining a loop, the free end of each of the leaf springs slidably extending through the band of the other leaf spring; and
said means for securing comprises a layer of adhesive disposed on each of the free ends adapted for attaching the free ends to opposing lateral edges of the ingrown toenail.

3. The self-adjusting tool for correcting an ingrown toenail according to claim 1, wherein:
said at least one elongate spring element comprises a single leaf spring having flat opposing ends; and
said means for securing comprises a first band and a second band, each of the bands defining a flat loop, and a layer of adhesive disposed on the bands adapted for attaching the bands to opposing lateral edges of the ingrown toenail, the opposing ends of the leaf spring slidably extending through the bands with the leaf spring extending across the toenail.

4. The self-adjusting tool for correcting an ingrown toenail according to claim 1, wherein:
said at least one elongate spring element comprises a single rod-shaped spring having opposing ends; and
said means for securing comprises a first cylindrical sleeve and a second cylindrical sleeve, and a layer of adhesive disposed on the sleeves adapted for attaching the sleeves to opposing lateral edges of the ingrown toenail, the opposing ends of the rod-shaped spring slidably extending through the sleeves with the rod-shaped spring extending across the toenail.

5. The self-adjusting tool for correcting an ingrown toenail according to claim 1, wherein:
said at least one elongate spring element comprises a plurality of rod-shaped springs having opposing ends; and
said means for securing comprises a pair of cylindrical sleeves corresponding to each of the rod-shaped springs, and a layer of adhesive disposed on the sleeves adapted for attaching the pairs of sleeves to opposing lateral edges of the ingrown toenail, the opposing ends of the rod-shaped springs slidably extending through the corresponding sleeves with the rod-shaped springs extending across the toenail.

6. The self-adjusting tool for correcting an ingrown toenail according to claim 1, wherein:
said at least one elongate spring element comprises a single planar T-shaped leaf spring having a crossbar and a shaft extending from the crossbar, the shaft having a flat end; and
said means for securing comprises a band defining a flat loop, and a layer of adhesive disposed on the band adapted for attaching the band to a lateral edge of the ingrown toenail and a second layer of adhesive disposed beneath the crossbar and adapted for securing the crossbar to an opposing lateral edge of the ingrown toenail, the flat end of the shaft of the leaf spring slidably extending through the band with the leaf spring extending across the toenail.

7. A method of correcting an ingrown toenail, comprising the step of anchoring spaced apart nodes of a resilient band tool to opposing lateral edges of the ingrown toenail with the tool flexed across the toenail so that the resilient band pulls the lateral edges of the ingrown toenail away from engagement with the toe by resilient force applied to the anchored nodes.

8. The method of correcting an ingrown toenail according to claim 7, further comprising the step of scraping the ingrown toenail until the toenail is thin prior to said step of anchoring the nodes.

9. The method of correcting an ingrown toenail according to claim 7, further comprising the step of shortening the ingrown toenail prior to said step of anchoring the nodes.

10. The method of correcting an ingrown toenail according to claim 7, further comprising the step of keeping the resilient band tool applied to the ingrown toenail for between one month and two months after the toenail has flattened in order to retract the edges of the toenail from the toe while any scars heal.

11. The method of correcting an ingrown toenail according to claim 7, wherein said the resilient band tool comprises at least one leaf spring.
